(19) United States
(12) Patent Application Publication
(10) Pub. No.: US 2013/0236445 A1
(43) Pub. Date: Sep. 12, 2013

(54) Bromelain proteinases (Bromelainases) as chemotherapeutic agents in treating and/or preventing various types of cancer in a mammal

(71) Applicants: Benedict Schue Liao, Los Angeles, CA (US); Alex B. Liao, Los Angeles, CA (US); Austin Lewis Liao, Los Angeles, CA (US); Judy Fu-Chuan Li-Liao, Los Angeles, CA (US); Burton Arthur Liao, Los Angeles, CA (US); Justin Liao, Irvine, CA (US); Edward Regean Liao, Irvine, CA (US); Robert Liao, Los Angeles, CA (US); Lily Liao, Los Angeles, CA (US); Susan Su-Hsin Tung-Liao, Iowa City, IA (US); Stanley Leo, Las Vegas, NV (US); Susan Joan Liao-Nieng, Chicago, IL (US); Su-Lien Liao-Chen, Baltimore, MD (US); Ludwig Schue Liao, Baltimore, MD (US)

(72) Inventors: Benedict Schue Liao, Los Angeles, CA (US); Alex B. Liao, Los Angeles, CA (US); Austin Lewis Liao, Los Angeles, CA (US); Judy Fu-Chuan Li-Liao, Los Angeles, CA (US); Burton Arthur Liao, Los Angeles, CA (US); Justin Liao, Irvine, CA (US); Edward Regean Liao, Irvine, CA (US); Robert Liao, Los Angeles, CA (US); Lily Liao, Los Angeles, CA (US); Susan Su-Hsin Tung-Liao, Iowa City, IA (US); Stanley Leo, Las Vegas, NV (US); Susan Joan Liao-Nieng, Chicago, IL (US); Su-Lien Liao-Chen, Baltimore, MD (US); Ludwig Schue Liao, Baltimore, MD (US)

(73) Assignee: Garfield Medical Group, Monterey Park, CA (US)

(21) Appl. No.: 13/374,328

(22) Filed: Dec. 16, 2011

(51) Int. Cl. A61K 38/48

(52) U.S. Cl. CPC A61K 38/4873
USPC 424/94.65

(57) ABSTRACT

A method for treating and/or preventing various types of human cancer and neoplastic diseases which is comprised of the administration thereto of an effective amount of Bromelain extract derived from Ananas Comosus and such proteins as ananase, bromelain, comosain, extranase, Inflamen, traumanase, and/or a mixture thereof.

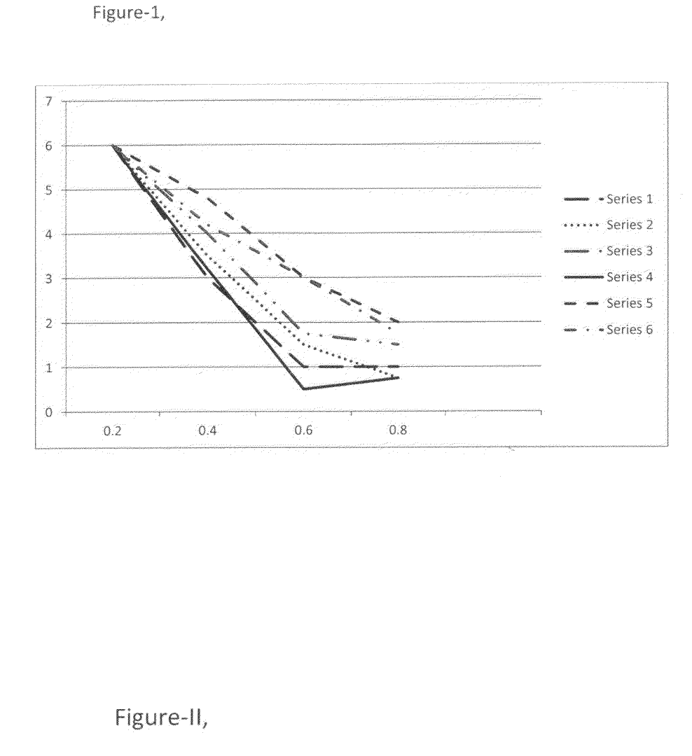

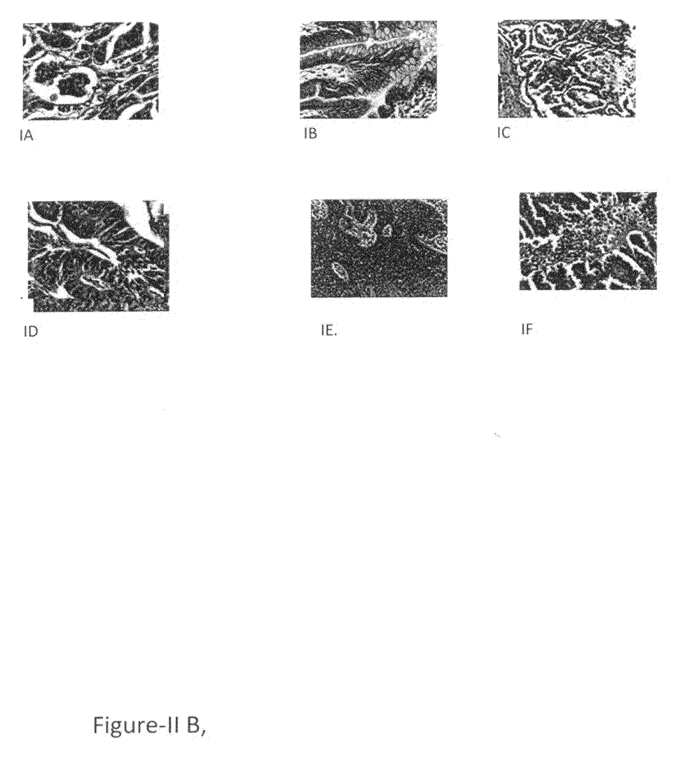

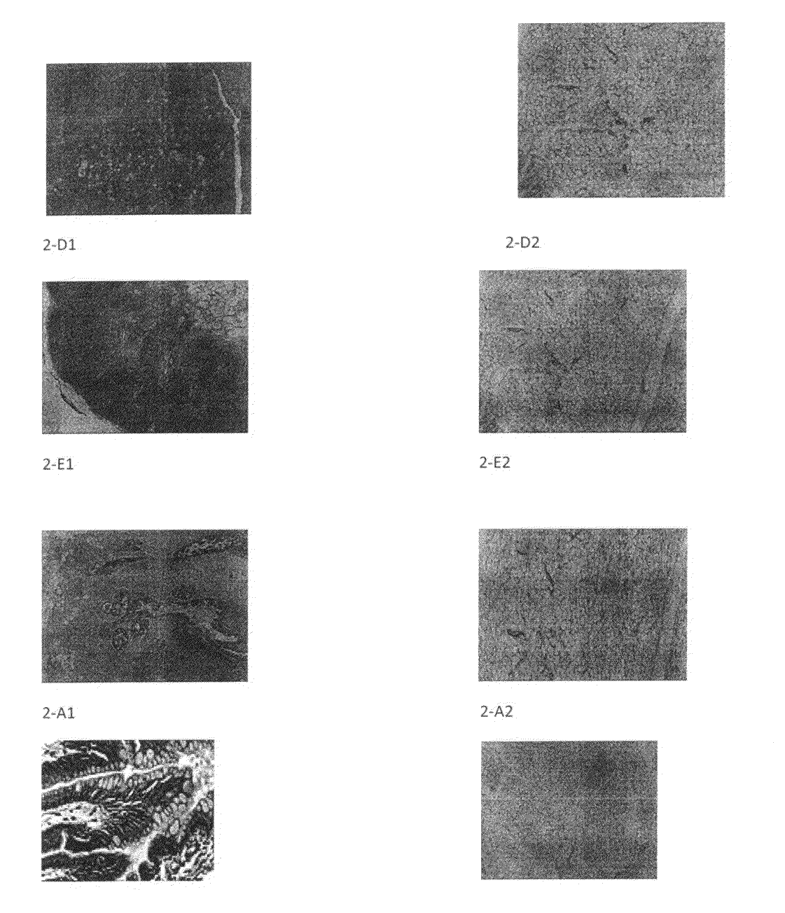

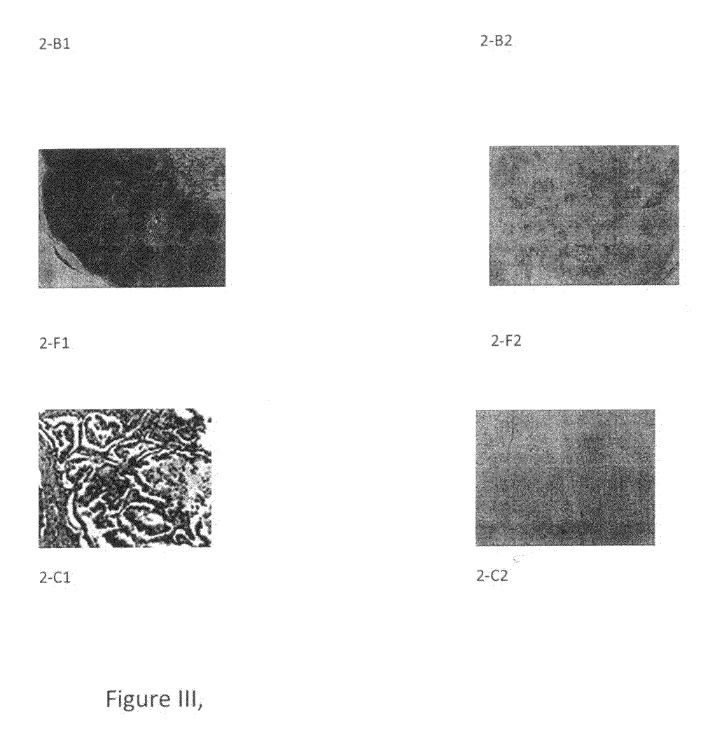

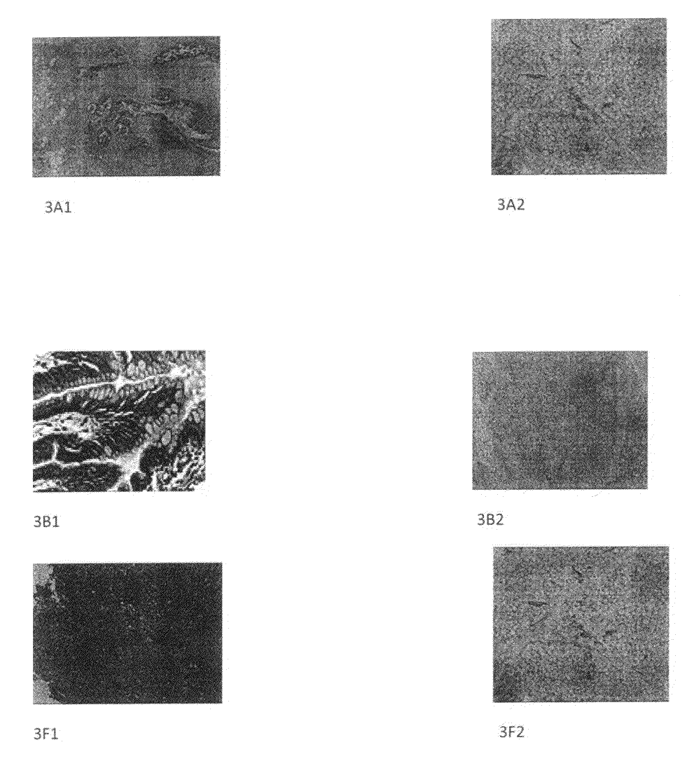

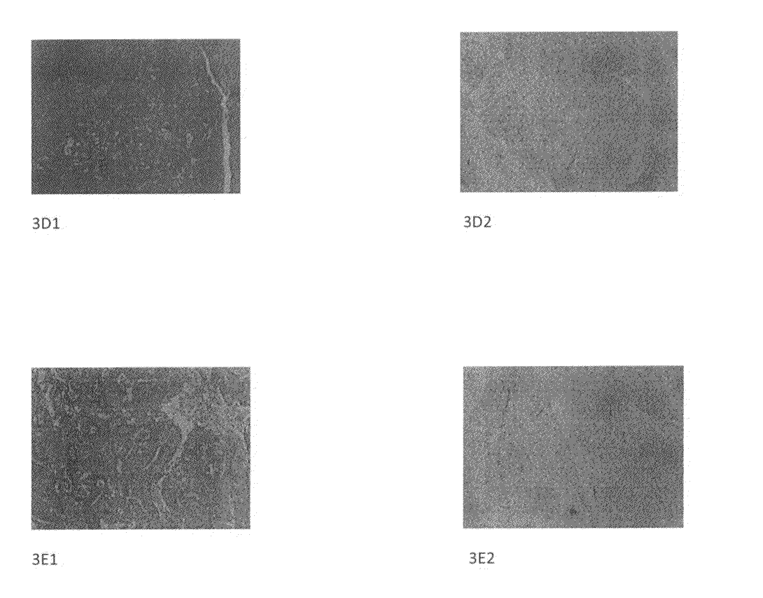

### CROSS REFERENCES TO THE RELATED APPLICATION

### References Cited

### U.S. Patent Documents

| Patent No. | Date | Inventors |
|---|---|---|
| U.S. Pat. No. 3,002,891 | October 1961 | Heinicke |
| U.S. Pat. No. 2,950,227 | August 1960 | Gibian, & Bratfisch. |
| U.S. Pat. No. 6,835,809 | December 2004 | Liu. et al |
| U.S. Pat. No. 3,691,016 | September 1972 | Patel |
| U.S. Pat. No. 4,247,642 | January 1981 | Hirohara, et al |
| U.S. Pat. No. 5,580,755 | December 1996 | Souza |
| U.S. Pat. No. 5,582,823 | December 1996 | Souza |
| U.S. Pat. No. 6,835,809 | December 2004 | Liu, and Feige et al |
| U.S. Pat. No. 7,335,382 | February 2008 | Ding, and Adrian et al |

### OTHER REFERENCES

1. Verma, A K.; Johnson, J A,; et al., Inhibition 7,12-Dimethylbenza-anthracene and N-Nitrosomethylurea-induced of mammary cancer by dietary favonoid Quercetin: Cancer Research: 48, pp. 5754-5758 (1988).
2. Castillo, M H.; and Perkins, E.; et al., The effect of Bio-flavonoid-Quercetin on squamous cell carcinoma of head and neck origin, American Journal of Surgery: 158, pp 351-355 (1989).
3. Singhal, R. L.; and Yeh, Y. A.; et al., Qucercetin down-regulates signal transduction in human breast cancer cells: Biochemistry, Biophysics Research Commun., 208, pp. 425-431. (1995).
4. Scambia, G.; and Ranelletti, F. O.; et al., Quercetin inhibits the growth of multidrug-resistant estrogen receptor-negative MCF-7 human breast cancer cell line expressing type II estrogen-bnding sites: Cancer Chemotherapy, Pharmacology: 28, pp. 255-258 (1991).
5. Scambia, G.; and Ranelletti. F. O.; et al., Type II estrogen binding sites in a lymphoblastoid cell line and growth-inhibitory effect of estrogen, anti-estrogen and bioflavonoids: International Journal of Cancer: 46: pp. 1112-1116 (1990).
6. Ranelletti, F. O.; and Ricci, R.; et al., Growth Inhibitory effect of quercetin and presence of Type-II estrogen-binding sites in human colon cancer cell lines and primary colorectal tumors; International Journal of Cancer: 50, pp. 486-492 (1992).
7. Peterson, G.; and Barnes, S.: Genistein Inhibition of the growth of human breast cancer cells: Independence from estrogen receptors and multi-drug resistance gene: Biochemistry, Biophysics Research Commun.: 179, pp. 661-667 (1991).
8. Barnes, S.; et al., Effect of genistein on in vitro and in vivo models of cancer: Journal of Nutrition: 125, pp. 7775-7835 (1995). Guthrie, N.; and Moffatt, M.; et al., Inhibition of proliferation of Human breast cancer cells by naringenin, a flavonoid in grapefruit: National Forum Breast Cancer, Montreal. P 119 (1993).
10. Kandaswami, C.; and Perkins, E.; et al., Anti-proliferative effects of citrus flavonoids on human squamous cell carcinoma in vitro: Cancer Lett.: 56, pp. 147-152 (1991).
11. Manach, C.; et al.; Bioavailability, metabolism, and physiological impact of 4-oxo-flavonoids: Nutrition Reseach 16: pp. 517-544 (1996).
12. Harrach, Tibor; and Eckert, Klaus; et al., Isolation and partial characterization of basic proteinases from stem bromelain: Journal of protein chemistry, Vol. 14, No. 1, pp. 41-52 (1995).
13. Grabowska, E.; Eckert, K; Fichiner, I.; Schultze-Forster, K.; and Maurer, H. R.: Bromelain proteases suppress growth, invasion and lung metastasis of B16F10 mouse melanoma cells. International Journal of Oncololgy; 11, pp. 243-248 (1997).
14. Harrach, T.; Eckert, K.; maurer H. R.; et al.; Isolation and characterization of two forms of an acidic bromelain proteinase; Journal of Protein Chemistry, 17, pp. 351-361 (1998).
15. Garbin, F.; Harrach, T.; Eckert, K.; and Maurer H R.: Bromelain proteinase F9 Augments human lymphocyte-mediated growth inhibition of various tumor cells in vitro, International Journal of Oncology 5: 197-203 (1994).
16. Harrach, T; Gebauer F.; Eckert, K; Kunze, R.; and Maurer H. R.; Bromelain proteinases modulate the CD44 expression on human Molt 4/8 leukemia and SK-Mel28 melanoma cells in vitro. International Journal of Oncology, 5: pp. 485-488 (1994).
17. Eckert, K.; Grunberg, E.; Garbin, F.; and Maurer H R.: Preclinical studies with prothymosin a-1 on mononuclear cells from tumor patients, International Journal Immuno pharmacology, 19: pp. 493-500 (1997).
18. Garbin, F.; Eckert, K.; Buttner, P.; Garbe C.; Maurer H R.: Prothymosin a-1 augments deficient antitumor activity of monocyte from melanoma patients in vitro; Anticancer Research 14, pp. 2405-2412 (1994).
19. Desser, L.; Rehberger, A.; Kokron, E.; and Paukovits, W.: Cytokine synthesis in human peripheral blood mononuclear cells after oral administration of polyenzyme preparation, Oncology 50, pp. 403-407 (1993).
20. Desser, L.; Rehberger, A.; and Paukovits, W.: Proteolytic enzymes and amylase induce cytokine production in human peripheral blood mononuclear cells in vitro, Cancer Biotherapy 9: pp. 253-263 (1994).
21 Van de Winkel, J G J.; Van Ommen, R.; et al., Proteolysis induces increased binding affinity of the moncyte type II FcR for human IgG, Journal of Immunology 143: pp. 571-578 (1989).
22. Batkin, S.; Taussig, S. J.; Szekerezes, J.; Antimetastatic effect of bromelain with or without its proteolytic and anticoagulant activity, Journal Cancer Res. Clinical Oncology. 114, pp. 507-508 (1988).
23. Hale, I.; and Haynes, B. F.: Bromelain treatment of human T cells removes CD44, CD45RA, E2/M1C2, CD6, CD7 and Leu 8/Lam1 surfaces molecules and markedly enhances CD2-mediated T cell activation: Journal of Immunology. 149, pp. 3809-3816 (1992).
24. Taussig, S. J.; Batkin, S.; and Szekerezes, J.: Inhibition of tumor growth in vitro by bromelain, an extract of the pineapple plant (ananas comosus), Planta Medica 6, pp. 538-539 (1985).
25. Ota, S.; Muta, E.; Katahira, Y.; and Okamoto, Y.: Reinvestigation of fractionation and some properties of the proteolytically active components of stem and fruit bromelain, Journal of Biochemistry, 98, pp. 219-228 (1985).
26. Rowan, A. D.; Buttle, D. J; and Barrett, A. J.: Ananain, A novel cysteine proteinase found in pineapple stem: Arch. Biochemistry Biophysics, 267, pp. 262-270 (1988).
27. Rowan, A. D.; Buttle, D. J.; and Barrett, A. J.: The cysteine proteinases of the pineapple plant, Biochemistry Journal, 266, pp. 869-875 (1990)
28. Yasuda, Y.; Takahashi, N.; and Murachi, T.; The composition and structure of carbohydrate moiety of stem bromelain, Biochemistry, 9, pp. 25-32 (1970).
29. Ishihara, H.; Takahashi, N.; Oguri, 5; and Tejima, S.: Complete structure of the carbohydrate moiety of stem bromelain, Journal of Biology and Chemistry, 254; 10715-10719 (1979).
30. Taussig, S. J.; Batkin, S.; Szekerezes, J.; et al.: Bromelain, the enzyme complex of pineapple (Ananas comosus) and its clinical application. An update, Journal of Ethnopharmacology, 22, pp. 191-203 (1988).
31. Kleef, R.; Delohery, T. M.; and Bovbjerg, D. J.; Selective modulation of cell adhesion molecules on lymphocytes by bromelain protease-5. Pathobiology, 64, pp. 339-346 (1996).
32. Mauer H. R.; Hozumi, M.; Honma, Y.; and Okabe-Kado, J.; Bromelain induces the differentiation of leukemic cells in vitro: an explanation for its cytostatic effect? Planta Medica, 54, pp. 377-381 (1988).
33. Heinckie, R M.; van der Wal L.; and Yokoyama, M.; effect of bromelain (ananase) on human platelet aggregation, Experientia 28: pp 844-845 (1971).
34. Matsumoto, G.; Nghiem, MP.; Nozaki, N.; Schmits, R.; and Penninger, J M., Cooperation between CD44 and LFA-1/CD11a adhesion receptors in lymphokine-Activated killer cell cytotoxicity, Journal of Immunology, 160, pp. 5781-589 (1998).
35. Mantovani, A.; Bottazzi, B.; Colotta, F.; Sozzani, S.; and Ruco, L.: The origin and function of tumor-associated macrophages. Immunology Today, 13, pp. 265-270 (1992).
36. Eckert, Klaus; Grabowska, Edyta; Strange, Rainer; Schneider, Ulrike; Maurer, H. Raine; et al.: Effects of oral bromelain administration on the impaired Immunocytoxicity of mononuclear cells from mammary tumor patients, Oncology Report, 6: 1191-1199 (1999).
37. Harrach T.; Eckert K.; Schulze-Forster K.; Nuck R.; Grunow D.; and Maurer H. Rainer: Isolation and partial characterization of basic proteinases from stem bromelain. Journal of Protein Chemistry 14: pp 41-52, (1995).
38. Harrach T.; Eckert K.; Schulze-Forster K.; Nuck R.; Grunow D.; and Maurer H. Rainer,; Machleidt I.; and Machleidt M.: Isolation and characterization of two forms of an acidic bromelain stem proteinase; Journal of Protein Chemistry 20; pp 53-64, (1997)
39. Taussig S J,; and Batkin S.; Bromelain, the enzyme complex of ananas comosus. Journal of Ethnopharmacology 22: pp 191-203, (1988)
40. Taussig S.; Batkin S.; and Szekerczes J.; antimetastatic effect of bromelain With or without its proteolytic and anticoagulant activity. Journal Cancer Research Clinical Oncology 114; pp 507-508, (1988).
41. Taussig S J.; Szekerczes J; and Batkin S.: Inhibition of tumor growth in vitro by bromelain, an extract of the pineapple plant (ananas comosus). Planta Medicus 6; pp 538-539, (1985).
42. Maurer H R.; Hozumi M.; Honma Y.; and Okabe-Kado J.; bromelain induces the differentiation of leukemic cells in vitro; an explanation for its cytostatic effect. Planta Medicus 54; pp 377-381, (1988).

### SUMMARY OF THE INVENTION

### Field of the Invention

The present discovery and invention relates to a method for preventing and/or treating the various types of cancer and/or neoplastic diseases. These include breast, colon, lung, ovarian cervical, uterine, and hepato-cellular carcinoma in a mammal, the method comprises administered an effective amount of Bromelain and its extract from Ananas Comosus.

### BACKGROUND OF THE INVENTION

According to the recent studies and reports, various types of cancer and neoplastic diseases have been the number one cause of deaths in America.

The search for new medicines with non-toxic, low side effects which are compatible with most other drugs has been under way since Cohen in 1964, Renzini in 1972, and Tinozzi in 1978. Bomelain was first isolated from pineapple fruit in 1891 by Marcano. (Marcano. Bull. Pharma. 5, 77 (1891). Heinecke and Gortner discovered stem bromelain, as a new proteinase preparation from pineapple plant in 1957, by precipitating with acetone and with ammonium sulfide in 1961. Further purification of crude preparation was formulated by Gibian and Bratfish in 1960 and a patent right was granted to Pineapple Research Institute (U.S. Pat. No. 3,002,891) and to AG Schering Company (U.S. Pat. No. 2,950,227). Bayer T., reported the anti-inflammatory effects in flavonoids (Phytochemistry, 28, pp 2373-2378 (1989) in 1989.

Utilization of plant and/or fruit flavonoids such as Rutin, Quercetin, Naringenin and Hesperetin in inhibiting tumor growth have been reported by Saija A., Scalese M. et al., (Free Radical Biology and Medicine vol. 19, no. 4 Pp 481-486 (1995)), Felicia, V. S.; Najla Guthrie, et al. depicted evidence of inhibition of human breast cancer cell proliferation and delay of mammary tumor-genesis by flavonoids and citrus juices. (Nutrition and Cancer vol. 26, no. 2, pp 167-181 (1996)).

In the year 2000, Revilla, E., and Ryan J. M., et al. analyzed of several phenolic compounds with potential antioxidant properties in grape extracts and wines by high-performance liquid chromatography-photodiode array detection without sample preparation. (Journal of Chromatographia; 6, 881 (1-2); pp 461-469 (2000)).

Also in 1991 by Kandaswai, C. and Perkins E. et al reported that citrus flavonoids have anti-proliferative effects on human squamous cell carcinoma in vitro: ((Cancer Lett.; 56, pp. 147-152 (1991)). Guthrie n., and Moffatt, M., et al claimed Naringenin, a flavonoid from grapefruit, has anti-proliferative effect in human breast cancer cell lines. (National Forum Breast Cancer, Montreal, p 119 (1993)).

The study of Rakotoarison, D A. et al showed anti-oxidant activity of polyphenolic extracts from flowers of Crataegus Monogyna (Pharmazie: 52: Pp 60-64 (1997)). Kaul, T N. and Elliott Middleton et al also reported the anti-viral effect of Citrus flavonoids on human viruses. (Journal of Medical Virology: 15; Pp 71-79 (1985)). It has also been reported that pant bio-flavonoids exhibit various biochemical and pharmacological activities including anti-oxidant, anti-inflammatory, anti-cancer, anti-viral and anti-platelet aggregation.

Nair, M G; and Wang, H B. et al reported that a method of inhibiting cyclooxygenase and inflammation using cyanidin), (U.S. patent Ser. No. 10/002,407)), Ranelleti, F. O., and Ricci R. et al, reported growth inhibitory effect of Quercetin and presence of type II estrogen binding sites in human colon cancer cell lines and primary colo-rectal tumors; (International Journal of Cancer: 50; pp 486-492 (1992)), Scambia, G. and Ranelleti, F. O. et al; reported that Quercetin inhibits the growth of a multi-drug-resistant estrogen receptor-negative MCF-7 human breast cancer cell line expressing type-II estrogen-binding sites: (Cancer Chemotherapy, Pharmacology: 28, pp 255-258 (1991)) also reported in Type II estrogen binding sites in a lymphoblast cell line and growth inhibiting effect of estrogen, anti-estrogen and bioflavonoids; (International Journal of Cancer: 46; pp 1112-1116 (1990)), Castillo, M. H. and Perkins, E., et al also reported the effect of the bioflavonoid-Quercetin on squamous cell carcinoma of head and neck origin; (American Journal of Surgery: 158, pp 351-355 (1989)).

Zhao, J., Wang, J., et al published a study showing anti-tumor promoting activity of a polyphenolic fraction isolated from grape seeds and identification of Procyanidin B 5-3′-gallate as the most effective antioxidant constituent; (Carcinogenesis, September; 20 (9); pp 1737-1745 (1999)).

In 1995 Harrach T., Eckert K., Schulze-Forster K. and Maurer H. Rainer et al reported isolation and partial characterization of basic proteinases from stem bromelain. (Journal Protein Chemistry 14: pp 41-52, 1995), and Again in 1997 they reported isolation and characterization of two forms of an acidic bromelain stem proteinase. (Journal Protein Chemistry 20: pp 53-64, 1997).

In 1985 Taussig, and Batkin et al suggested that the enzyme complex from Ananas Comosus (pineapple) produced inhibition of tumor growth in vitro (Planta Med. 6: 538-539, 1985). and outlined its clinical application, (Journal Ethnopharmacology: 22, P 191-203, 1988) The same enzyme complex also produces platelet anti-aggregation, and fibrinolytic activity, (reported by Marz, in 1982, Heinecke in 1972, Heinecke and Yokoyama in 1957). In 1979 Klaue, Amen, and Roman et aI reported use of bromelain as chemical debridement agent in 3^{rd} degree burn patients. (European Surgical research, 1979, PP 355-359). In same year of 1988 the same team also discovered that Bromelain has an anti-metastatic effect with or without its proteolytic and anti-coagulant activity, (Journal of Cancer Research Clinical Oncoloy, 114: pp 507-508, 1988).

The present inventors have discovered that the extract of Bromelain proteins which derived from the fruit and stem of Ananas Comosus (pineapple). (Bromelian, Ananase, Inflamen, Extranase, Traumanase) which are effective in treating and/or preventing various types of cancer and neoplastic diseases which including breast, colon, lung, ovarian, cervical; and uterine cancers, by

(a) its anti-inflammatory properties,
(b) anti-platelet aggregation (Mynott et al in 1998, suggested that it changes the tumor surface antigen thus preventing tumor cells from attacking the normal tissues),
(c) anti-tumor genesis; Mynott et al in 1998 suggested this action was probably due to release of tumor necrotizing factors (TNF) in T-cells of WBC. Taussig et al in 1985 and Taussig, and Batkin et al in 1988 both indicated that Bromelain extract can be used in inhibiting tumor growth) and The T-cells, peripherial blood mononuclear lymphocytes (PMN) with influence of bromelain, they produce and out pour of TCRS/CD2, TCRS/CD3 which induce massive amount of Interlukin IB, II-6, II-8, and TNF, and attack the tumor surface antigen of CD44, CD45, CD47. The mechanism of this stimulation are through two pathways (1) Major Mitogen Activating Protein Kinases (MMAP) (2) The second pathway is through the engagement of Bromelain (Antigen Polypeptide) (APP) presented by the Major HistoCompatibility Complex (MHC) expressed on Antigen Presenting Cells (APC) (Cantrell 1996).
(d) anti-dedifferentiation both in vitro and in vivo in cancer cell lines, animal, and human experiments.
(e) Inhibit tumor cell growth, mainly due to tumor necrotizing factors (TNF). Also channel through TCRS/CD2, and TCRS/CD3 which induce massive amount of secretion of TNF. About 42 folds of normal production (Yoshihara 1996, and Maurer 1985 and 1988)

### SUMMARY OF THE INVENTION

The present invention and discovery relates to a method for treating and for preventing various types of cancerous and neoplastic disorders in mammals, which comprises the administration thereto of an effective amount of Bromelain extract derived from the stem and fruit of Ananas Comosus, such as bromelain; ananase; comosain, extranase; inflamen; traumanase; or a mixture thereof.

### DETAILED DESCRIPTION OF THE PREFERRED EMBODIMENTS

### Detailed Description of the Invention and Drawing

Bromelain extract including both stem and fruit bromelains, have all the effects that natural bromelains depict including anti-inflammation, anti-platelet aggregation, fibrinolytic properties, anti-tumor growth, and differentiation effect in tumor cells. Fruit bromelain from pineapple was first isolated by Marcano in 1891 (Marcino Bull. Pharm. 5, 77 (1891), and from pineapple juice by precipitation with acetone and also with ammonium sulfide: Heinecke in 1961 (U.S. Pat. No. 3,002,891). Bromelain has a molecular weight of 33,000. It is a glycoprotein, was purified from crude preparations by Gibian, and Bratfisch et al in 1960 (U.S. Pat. No. 2,950,227).

Stem bromelain was discovered by Balls et al in 1941, (Ind. Eng. Chem. 33; 950, 1941), both fruit and stem bromelains are acidic and basic proteins with ultraviolet light wavelength of 280 nm (A 1%/1 cm 20.1)

### BRIEF DESCRIPTION OF THE DRAWINGS AND FIGURES

The objects of the present invention will become increasingly apparent by reference to the following descriptions and drawings.

Table I, Overview of the Bromelains that can be used in these characterized cells.

CMLS, Cell. Mol. Life Sci. Vol. 58, 2001 Review Article

TABLE 3
Bromelain effects, in vitro (BP, F4, F5, F9) and in vivo (POS), on tumor cell growth and metastasis.

| Protease | Parameter | Effect | Signifiance | [symbol] |
|---|---|---|---|---|
| Bromelain BP | tumor cell proliferation | ↓ | reduction of tumor cell growth | [symbol] |
| Bromelain F9 | | | | [symbol] |
| Bromelain BP | tumor cell invasion through | ↓ | reduction of the invasive potential of | [symbol] |
| Bromelain F4, F9 | extracellular matrix | = | rumor cells | |
| Bromelain F5 | | | | |
| Bromelain POS | growth of lung metastases in mice | ↓ | reduction of the metastatic potential of | [symbol] |
| | | | tumor cells | |
| Bromelain BP | CD44 expression on metastatic cells | ↓ | reduction of tumor cell adhesion to | [symbol] |
| Bromelain F9 | | | endothelial cells | [symbol] |

[symbol]

indicates data missing or illegible when filed

TABLE 1
Cysteine proteinases (bromelains) from pineapples (Ananas comosus).

| Name | | | | | |
|---|---|---|---|---|---|
| (EC number) | Abbreviation | | | | |
| accordimg | according to | Molecular mass | Isoelectric | | |
| to [2, 8] | [6, 7] | (Dalton) | point | Sequences | Glycosylation |

From pineapple stems:

| | | | | | |
|---|---|---|---|---|---|
| Stem bromelain | F4 and F5 | 23,800 | >10 | completely | glycosylated |
| (EC 3.4.22.32) | | (sequence + sugar) | | sequenced | |
| | | | | (212 amino acids) | |
| Ananain | F9 | 23,464 | >10 | completely | not glycosylated |
| (EC 3.4.22.31) | | (sequence) | | sequenced | |
| | | | | (216 amino acids) | |
| Comosain | F9/b | 24,509 and 23,569 | >10 | N-term, sequence | glycosylated |
| | | (esms) | | | |
| | SBA/a and SBA/b | 23,550 and 23,560 | 4.8 and 4.9 | N-term, sequence | highly [symbol] |

[symbol]

indicates data missing or illegible when filed

TABLE 4
Immune cell mediated bromelain effects, in vitro (by bromelain
F9) and in vivo (by bromelain POS), against tumor cells.

| Protease | Parameter | Effect | Significance | Ref. |
|---|---|---|---|---|
| Bromelain F9 | immunocytotoxicity of lymphocytes | ↑ | increase of cellular immune response | 45 |
| | against tumor cells | | | |
| Bromelain F9 | secretion of TNFα, II-2 | ↑ | stimulation of lymphocytes | 45 |
| Bromelain POS | immunocytotoxicity of lymphocytes, | = | cellular immune response in vivo | 39 |
| | monocytes from tumor patients | ↑ | | |
| | against tumor cells in vivo | | | |
| Bromelain POS | secretion of II-1β from human | ↑ | reduction of tumor cell adhesion to | 33 |
| | monocytes | | endothelial cells | |
| Bromelain POS | expression of cell surface markers on | ↓ | regulation of cell adhesion and signal | 33 |
| | lymphocytes from tumor patients | | transduction of lymphocytes | |
| | CD44 | | | |

Six types of cancer cell lines were used in our present invention and discovery, which included breast, lung, colon, cervical, ovarian, and uterine cancers. Most of our cancer cell lines were harvested directly from surgical specimens during surgery, and were prepared and described in detail under cell culture in Example-2.

Table II, Detail information (Supplier and Catalogue numbers) from bromelain proteins and proteinase molecules. The stem bromelain was purchased from Sigma-Aldrich Co. St. Louis, Mo. (Catalogue No#4882), Complete Growth Medium (Catalogue #M4655), Tween-20, and Tween-80 solution (Catalogue No #P2287, #P 8192), Penicillin-streptomycin-Neomycin Stabilized Solution (Catalogue No#P 4083), New born calf serum (Catalogue No #4762). Cell culture wares were from Becton-Dickinson Co, Franklin Lakes, N.J., (Catalogue No #353503,), B-D TM Cell viablility kit with liquid counting beads (Catalogue No #349486), B-D FACS Array tm Bioanaalyzer (Catalogue No #340128). Pathological and microscopic images were taken by Amescope Trinocular Microscope (American Optic Co., Model No#T-490B-10MA).

The complete growth medium (CGM): consists of Dulbecco's modified essential medium (Sigma-Aldrich Co., St. Louis, Mo.), supplemented with 10% heat inactivated new born calf serum, 2% L-glutamine, penicillin (100 iu/ml), streptomycin (5 mg/ml), and neomycin (10 mg/ml) (also from Sigma-Aldrich Co. St. Louis, Mo.). The cells were maintained in a standard tissue culture incubator at 37 degree C. with atmosphere humidity of 90% air, and 10% CO 2. All cancer cell lines were initiated by seeding 5×(10) 6 cells into 75 cmsq tissue culture flasks and base were coated with 0.75% agar in CGM. The cancer cell lines were used between 5 and 7 days of culture.

figure-1, depicted Growth inhibition of various types of tumor cell lines in vitro. Growth inhibition of various types of tumor cell lines in vitro. Bromelain in medium in mg/ml in X-axis, percent (%) of cell growth VS control in Y-axis.

Inhibition of tumor cell growth with increasing Bromelain concentration in all six cancer lines: breast, colon, lung, ovarian, cervical, and uterine cancers. The cells in all groups were incubated for 72 hours at 37 C degree in 10% CO2 in air and were counted with Coulter counter.

The graph represents the six individual experiments with 6 tumor cell cultures/each tumor line in each experiment.

Figure-II, Depicts the pre-treatment pathological pictures of tumor cells In various types of cancer in experimental animal, and human models
I-A (breast cancer) I-B (colon cancer); I-C (lung cancer);
I-D (Ovarian cancer) I-E (Cervical cancer) I-F (Uterine cancer)

Figure-III, Depicts the post-treatment pathological pictures of the tumor cells in various types of cancer in experimental animal models which were treated with intra-peritoneal injection of bromelain In an amount of 25.0 mg/twice a week for 8 weeks
2A (breast cancer); 2B (colon cancer); 2C (lung cancer);
2D (ovarian cancer); 2E (cervical cancer); 2F (uterine cancer).
   all cancer cells after 8 weeks of bromelain treatment, the pathological pictures all showed fibrosis of the tissues.

Figure III, Depicts the post-treatment by either CT scan report and/or pathological pictures in various types of cancer in human model which were treated with bromelain by oral and intravenous infusion. The pathological findings are all fibrosis.
3A (breast cancer); 3B (colon cancer); 3C (lung cancer);
3D (ovarian cancer); 3E (cervical cancer); 3F (uterine cancer).
3-A represents Breast cancer cells after bromelain treatment for 6 months.
3-B represent colon cancer cells after bromelain treatment for 6 months.
3-C represents lung cancer cells after bromelain treatment for 6 months.
3-D represents ovarian cancer cells after bromelain treatment for 6 months.
3-E represents cervical cancer cells after bromelain treatment for 6 months.
3-F represents Uterine cancer cells after received bromelain treatment for 6 months

### PREPARATION AND ANALYSIS OF BROMELAIN EXTRACT

### Example-A

### Bromelain Extraction Procedure

The pineapple stumps were stripped of leaves and roots and outer epidermal layers. They were then cleaned and fed to a sugar mill press, the residues were returned to the mill press for an additional three times, after the second run, water was added to increase the efficiency of the extraction process. The juice produced was about 35% of peeled pineapple stumps. The protein produced was 150 grams per 10 pounds of peeled stumps. The total yield of proteinases, expressed in milk clotting units (M.C.U.) was 25,000 units per pound of stump.

### Example-B

### Bromelain Precipitation by Acetone

One liter of cold clarified stem juice at pH 5.0 was added to 370 ml of cold acetone. both at a temperature of 34 degrees F. The mixture was held for an hour and the supernatant containing the desired enzyme substances was syphoned off. The precipitate contained about one-sixth of the activity of the final product. After all assays on this fraction were run, it was discarded. Again 630 ml of cold acetone at 34 degree F. was added to the combined supernatant, the precipitate was collected by centrifuging of the combination supernatant. The enzyme was left in the centrifuge bottle and dried in a vacuum chamber at a low temperature. The yield of enzyme was 9.5 grams of a white powder which contained 4500 M.C.U./per gram. based on the activity of the starting juice, which represented 40% of the proteolytic activity originally present in the juice.

### Example-C

### Bromelain Precipitation by Ammonium Sulfate

1000 ml of cold (40 degree F.) clarified pineapple stem juice at PH of 4.0 were added 210 gram of ammonium sulfate. The precipitate formed was removed by centrifuging. Another 150 gram of ammonium sulfate was added to the supernatant. The precipitate was collected by centrifuging. Then dissolved in water and dialyzed to remove the ammonium sulfate. The enzyme was recovered from the salt free solution by acetone precipitation. The total yield was 115 mg of precipitates of protein with activity of 2200 M.C.U./gram.

### Example-D

### Bromelain Precipitation by Dialysis

2000 ml of the freshly pressed juice was placed in a web-cell dialyzed chamber, and was dialyzed against running tap water at 3 degree C. for two days and then dialyzed against five gallons of distilled water for one day. During the dialysis a light grey precipitate appeared was removed and discarded. The supernatant was then freeze dried and yield of 9.6 grams of 4200M.C.U. enzyme per liter.

### Example-1

### Toxicity of Bromelain in Mice by Oral Administration

30 specimens of 8 week old, with specific pathogen free, ICR female mice, each weighing 25-30 gram, were divided into five groups (6 mice each) and were kept in %, and 12 H light/12 H dark photoperiod, they were fed with Harlan Teklad-2018 global rodent diet (18% protein) (Kaytee Co. Madison, Wis. USA); drinking water was sterilized.

Bromelain (Aldrich-Sigma Co. St. Louis, Mo. USA) was dissolved in 0.5% of tween-80 solution to a final concentration of 50 mg/ml, 100 mg/ml, 125 mg/ml and 150 mg/ml respectively, and was orally fed to the 4 separated groups of mice in an amount of 0.2 ml per 20 gram of mouse body weight, that is contains 500 mg/kg, 1000 mg/kg, 1250 mg/kg, and 1500 mg/kg separately.

One group of 6 mice was kept as control group and was not fed with the bromelain solution. The solution was administered once every 6 months, and the mice were observed for 18 months for the signs of adverse effects or death according the following schedule: 1 H, 4 H, 8 H, 12 H (hour), after administration and then every 12 hours thereafter. The daily weight of each mouse was recorded. On day 546, (18 months later) the mice were sacrificed, and the internal organs including liver, kidney, heart, lung, muscle, stomach, urinary bladder, intestines, pancreas, and spleen were examined visually and microscopically.

All mice were alive at 18 months, and no body weight loss occurred during this period of observation. The mice did not develop any pathological abnormality either visually or microscopically. Therefore, it is concluded that Bromelain extract from pineapple is not toxic

### Example-2

### Bromelain Inhibit Cancer Cell Growth In Vitro

Cancer cell lines were developed either from direct harvest from surgical specimens during surgery or from friends at different oncology institutes. The specimens were emulsified in normal saline and filtrated three times with the mesh permitting less than 5 um particles to pass through. The supernatants were preserved in Complete Growth Medium (CGM)) (Sigma-Aldrich Co. St. Louis, Mo.) in the 75 ml flasks at 8 degree C. until they are ready to be used for seeding. in a standard tissue culture. Complete Growth Medium (CGM) consisting of: Dulbecco's modified Essential medium (Sigma-Aldrich Co. St. Louis, Mo.) supplemented with 10% heat inactivated new born calf serum, and 2% L-glutamine, penicillin (100 IU/ml), streptomycin (5 mg/ml), and neomycin (10 mg/ml) (all from Sigma-Aldrich Co. St. Louis, Mo.) and incubated at 37 degree C. with humidified atmosphere of 90% air, 10% CO2 and cell split at 1:2 rate twice weekly.

Six various types of cancer cell lines which included breast, lung, colon, cervical, uterine, and ovarian cancer were used to test the sensitivity of the growth inhibition by the Bromelain in different concentrations. The cells were seeded in 2×(10)3 into 96 wells tissue culture microtiter plates (Becton—Dickinson Co. Franklin Lakes, N.J.). The cells were maintained in a standard tissue culture In Complete Growth Medium (CGM). Bromelain solutions were added to culture media in the following concentration: 0.2 mg/ml, 0.4 mg/ml, 0.6 mg/ml, and 0.8 mg/ml for 72 hours and tumor cells were counted with Coulter counter Model B, Beckman Coulter, Co.). The tumor cell growth inhibition percentages are depicted in Figure-1. which demonstrates that the growth of all cancer cell lines were inhibited in higher concentrations of Bromelain.

Inhibition of tumor cell growth with increasing Bromelain concentration in all six cancer lines: breast, colon, lung, ovarian, cervical, and uterine cancers. The cells in all groups were incubated for 72 hours at 37 C degree in 10% CO2 in air and were counted with Coulter counter. The graph represents the six individual experiments with 6 tumor cell cultures/each tumor line in each experiment. Please see Figure-I;

Line series-1 represents breast cancer cell lines;
line series-2, represents lung cancer cell lines;
line series 3, represents colon cancer cell lines;
line series-5 represents cervical cancer cell lines;
line series-6 represents uterine cancer cell lines.

All cancer cell lines showed 50% growth inhibition in the concentration of bromelain at 0.6 mg/ml, and achieved 80-90% tumorcidal effect at 1 mg/ml concentration of bromelain solution.

### Example-3

### Bromelain Inhibits Cancer Cell Growth In Vivo on Experimental Animal

### Bromelain Intraperitoneal Administration to Experimental Animal.

14 Experimental animals of 4 to 6 weeks old white rabbits each weighting 1 to 1½ pound were fed under a condition of 23+_{—}3 degree C., relative humidity of 45+_{—}5% and photoperiod of 12 light/12 dark. The rabbits were divided into seven groups with 2 heads each and were fed with Harlan-Taklad rabbit diet TD-1376 (Madison, Wis.) containing moisture 12%, crude protein 16%, crude fat 2%, crude fiber 15%, ash 8%, and 47% of nitrogen free Substances.

The rabbits were fed for 3 weeks with free access to the diet and water. Body weight was recorded every 7 days, and records were analyzed. All rabbits showed a normal growth rate with no significant differences among the seven groups in regard to the diet ingestion amount or the body weight gain.

The cancer cell lines were injected into six groups of the rabbits, that is 2 heads each group with 2 heads served as control (without tumor cells injection). The cancer cell lines were developed from Example-2. Each head was injected 0.5 ml of different cell line fluid intraperitoneally, prefer in the peritoneum layer, then the rabbits were fed the same diet for 3-4 weeks until a tumor grew in the peritoneum. The size, and location of the tumors were recorded.

When the tumors reached to 3-5 mm diameter in size. Bromelain in the amount of 25.0 mg/ml in normal saline with 100 mg of vitamin C (to keep the solution acidified), one ml of bromelain was injected into six different group of rabbits, the bromelain were given twice a week for 8 weeks,

After 8 weeks of treatment, the rabbits were anesthetized with injections of ketamine 75 mg/kg in the femoral muscle and sacrificed. Blood samples were collected from the heart of each rabbit to determine the blood analysis consisting of: complete blood count (CBC), Chemistry-7 and 24, (including Liver and renal function tests), lipid profiles (including Total cholesterol, HDL, LDL, VLDL, and triglycerides), coagulation factors consisting of; prothrombin time (PT), partial thromboplastin time (PPT), and immune-globulin-E.

All the laboratory tests were analyzed, and showed no differences among or within each groups. All laboratory blood analysis were performed on rabbits of all 7 groups, The results were tested using student t-test and Microsoft Excel-7 programs. The results are depicted in Table-II.

Table-II, The table presents blood analysis of the rabbits of 6 different groups which were treated with bromelain after inoculation of cancer cell lines.

Control group; TC (183.3+_{—}50.2 mg/dl), TRG (110+_{—}40.6 mg/dl), HDL (45.6+_{—}20.4 mg/dl), SGOT (38.6+_{—}6.2 u/l), SGPT (62.5+_{—}6.5 u/l),
GGTP (8+_{—}2.4 u/l), WBC (6.8+_{—}2.0 k/ul), Hb; (12.3+_{—}2.2 gm/dl).
Bromelain treated groups; TC (175.6+_{—}36.8 mg/dl), TRG (92.6+_{—}38.8 mg/dl), HDL (43.6+_{—}16.5 mg/), SGOT (110.8+_{—}30.7 u/l), SGPT (71.2+_{—}3.8 u/l), GGTP (7+_{—}1), WBC (7.3+_{—}2.2 k/ul), Hb; (11.9+_{—}1.9 gm/dl).

### TC; Total Cholesterol, TRG: Triglycerides, WBC: White Blood Cell, HDL; High Density Lipoprotein, SGOT; Serum Glutamo-Oxalic Transferase, SGPT; Serum Glutamo-Pyruvic Transferase, Hb; Hemoglobin.

The internal organs from the rabbits sacrified in this sample including lung, heart, liver, kidney, muscle, omentum, intestine, stomach bladder and pancreas were visual examined and showed no abnormalities. One half of each organ was frozen and the other half was fixed with 10% neutral buffered formalin for 24 hours. Then the fixed organs were washed with tap waterand stepwise dehydrated with 70%, 80%, 90%, 100% ethanol and then embedded in paraffin by using Shandon-Histocentre-2. The embedded organ blocks were sectioned in 4 mu.m thickness with a microtome (McBain, M 820, American Optical Co. USA) and stained with hematoxylin and eosin stain (H.E. stain). The stained specimens were made transparent with xylene, and mounted with permount on microslides. There were no pathological abnormalities or lesions under microscopic examination.

All the specimens collected from six group of rabbits showed no evidence of persistent disease or cancer cells.

Therefore, we suggest that Bromelain can served as a chemotherapeutic agent in various types of cancer in experimental animal without side effects.

### Example-4

### Bromelain Oral Administration to Inhibit Tumor Growth in Humans

### Bromelain Oral Administration to Late Stage Cancer Patients

24 volunteers were divided into six groups (four in each group) and 6 persons serve as control group (no bromelain treatment). All were in their 4th and 6th decades with various types of cancers including breast, lung, colon, ovarian, cervical and uterine origins. All were in either Stage III or Stage IV (the cancers had metastase widely either to lung, liver, bladder or rectum). All had been treated with either radiation or chemotherapy after surgery but experienced no positive results. The bromelain were administered in doses of 20-30 mg/kg based on 50-60 kg of body weight. That is 1000 mg-to-1500 mg/day divided into two doses. Patients were monitored with bi-weekly blood tests consisting complete blood counts, chemistry-7, chemistry-24, kidney and liver function tests, tumor markers, coagulation factors, and X-ray or CT scan in appropriate areas to determine the size of the tumors. There were no abnormalities in all the blood tests, no anemia, no leucopenia, no thromcytopenia, no abnormal kidney nor liver function tests. Tumor markers decreased, tumors were shrank and decreased in size on the X-ray or CT scan measurement. Patients' lifestyle become manageable, and improved considerably. The treatment period were varied from 12 to 24 months. At the end of the treatment in the treated groups no patients expired, However, all patients in control group, who had not wish to be treated, succumbed to their related cancers in 6-12 months.

(Step-A) After blood samples were collected and allowed to stand for 2 hours, then centrifuged at 4000 rpm for 10 minutes. (Megafuge, Baxter-Heraeus Instrument Co. N.J.). The superantants were separated and stored in a deep freeze before analysis. The chemistry analysis was carried out by blood chemistry analyzer (Cobra-Integra-700, Roche Diagnostic Lab. Indiana.) to determine the changes in total cholesterol, HDL, LDL, triglycerides, liver function tests (such as SGOT, SGPT, G-GPT), renal function tests, and coagulation factors (PT, PTT)(Bayer-MLA-Electra-900 Automatic coagulation Timer). All results were tested with student t-test and Microsoft Excel-7.0 program. The results are depicted in Table-III. Which Table-III, This table presents blood analysis of 24 volunteers suffering from various types of cancer with bromelain oral therapy.

TC; (210.3+_{—}30.2 mg/di), TRG; (165.5+_{—}28.3 mg/dl), HDL; (43.3+_{—}22.2 mg/dl), SGOT; (34.7+_{—}6.2 u/l), SGPT; (63.3+_{—}5.6 u/l), GGTP; (7.2+_{—}2.1 u/l)
WBC; (6.7+_{—}2.8 k/ul), Hb; (12.3+_{—}2.1 gm/dl)

Therefore, we concluded that the treatment of these various represented types of cancers with large doses and prolong periods with BROMELAIN are effective and without side effects.

### Example-5

### Bromelain Intravenous Administration to Inhibit Tumor Growth in Humans

In addition to a control group of two untreated individuals, tested groups were established as follows; twelve human volunteers age from 4th through 6 decades, were divided into six groups of two, all of whom were in Stage-3, and Stage-4 (as described above in Example-4) cancers of breast, colon, lung, ovary, cervix, and uterus. In the past, All had chemotherapy, and/or radiation therapy after surgery without effect. They were given Bromelain in doses of 50 to 60 mg/kg, body weight of 50 to 60 kg, which equivalent of 2000 to 3000 mg each week/in two divided doses, Bromelain was administered intravenously in 3-4 hours period, for the 12 weeks-to 2 years. In addition to the Intravenous infusion of bromelain, all patients in the test groups also received oral bromelain in doses of 50 mg/kg/day to increase therapeutic effects.

Blood tests, including CBC, Chemistry-7, and 24, liver and renal function tests, lipid profile, coagulation factors, PT., PTT, IGG, tumor markers were tested bi-weekly. X-ray, and CT scan were performed to measure the size of the tumors.

The blood analysis on these groups of patients showed no affects from the bromelain infusion treatment. (The results are not depicted.)

At the conclusion of the treatment period, the results are depicted as follows:

### (A) Two Breast Cancer Patients;

(1). One patient experienced left breast tumor shrinkage from 10.5 cm×8.4 cm to 2.5 cm×1.8 cm in size. The left axillary lymph node shrank from 2.5 cm×1.6 cm to 0.5 cm×0.4 cm (Left breast). with 12 weeks of intra-venous (IV) bromelain therapy.
(2) The second patient's right breast tumor shrank from 6.5 cm×4.8 cm to 2.8 cm×1.5 cm. with 16 weeks of intravenous bromelain therapy.

### (B) Two Lung Cancer Patients;

(1) One patient experienced the shrinkage from 4.6 cm×5.1 cm to 1.8 cm×1.2 cm with 18 weeks of intra-venous bromelain therapy.
(2) Another patient with lung tumor of 3.9 cm×4.5 cm experienced reduction of the tumor to 1.5 cm×1.9 cm with 20 weeks of IV bromelain therapy.

### (C) Two Colon Cancer Patients;

(1) Two patients with stage-IV disease and widely metastasis in the abdominal cavity, have been treated with intra-venous bromelain infusion for 18 months, and now show no evidence of persistent disease.

### (D) Two Ovarian Cancer Patients;

Both patients suffered stage-III C with wide intra-abdominal metastasis, both had been treated with intra-venous bromelain infusion for 18 months, and showed no evidence of persistent disease on CT Scan reports.

(E). Two Patients with Cervical Cancers;

Both patients were stage-IV with rectal and urinary bladder invasions. After intense bromelain intra-venous treatment for 12 months, there was no further evidence bladder or rectal invasion. No tumors could be detected.

(F). Two Patients with Uterine Cancer;

Both patients were Stage-III with intra-vaginal metastasis, After intra-venous bromelain treatment for 12 months, tumors in the vagina showed necrosis and fibrosis with no evidence of persistent disease.

### (G). Two Patients Comprising Control Group Who Refused to be Treated;

One patient with cervical cancer stage-IV with cervical spine metastasis, and one patient with breast cancer stage-IV with pulmonary metastasis. Both patients refused treatment, and both succumbed to their diseases with pulmonary, and septic infections in 9 to 12 months after frequent in-hospital cares and treatments.

Again, this is evidence that administration of Bromelain extract (Bromelainases) in humans could treat various types of cancer and neoplastic diseases. Liver and renal function, lipid profile, white blood cells and hemoglobin were not affected.

### Example-6

### Pharmaceutical Formulation and Preparation

Hard and/or soft gelatin capsules are prepared with ingredients as follows:

Formulation-1

Quantity (mg/capsule): Active ingredient 250, Vitamin C 200, Starch or Lactose (carrier) 50, Total 500 mg.

Formulation-2

Quantity (mg/capsule): Active ingredient 500, Vitamin C 300, Starch or Lactose (carrier) 200, Total 1000 mg.

Formulation-3

Quantity (mg/capsule): Active ingredient 750, Vitamin C 100, Starch or Lactose (carrier) 150, Total 1000 mg.

While the present invention and discovery has been described with respect to the above specific descriptions and embodiments, it should be recognized that various modifications and changes may be made to the present invention by those skills in the art which also fall within the scope of the invention as defined by the appended claims and their legal equivalents.

What is claimed:

1. The present discovery and invention relates to a method for treating and/or preventing various types of cancerous disease in a mammal, which is comprised of the administration thereto of an effective amount of Bromelain proteins, enzyme complex derived from Ananas Comosus.

2. The method of claim 1: wherein the mammal is human.

3. The method of claim 1: Wherein the bromelain proteinases mixture thereof is administered in the form of pharmaceutical composition containing an effective amount of bromelain and pharmaceutically acceptable excipients, carriers or dilutents.

4. The method of claim 1, wherein the effective amount of the bromelain proteinases or a mixture thereof ranges from 0.1 to 500 mg/kg of body weight/day.

5. The method of claim 1, the bromelain proteins derived from Ananas Comosus and with a solvent selected from the group consisting of water, a low alcohol and an aqueous alkali- or alkaline earth-metal hydroxide solution and/or an acid solution.

6. The method of claim 3, wherein the bromelain proteins have cytotoxic (through T-cell), anti-platelet aggregation (anti-metastasis effect), anti-inflammation, and anti-tumor-genesis (anti-proliferation and tumor necrotizing factor) effect against various types of cancer cell lines, in animal and human experiments both in vitro and in vivo, such as breast, colon, lung, ovarian, cervical, and uterine cancer.
